(19) United States
(12) Patent Application Publication
(10) Pub. No.: US 2011/0287057 A1
(43) Pub. Date: Nov. 24, 2011

(54) Cancer Treatment

(75) Inventor: ECKHARD R. PODACK, Coconut Grove, FL (US)

(73) Assignee: UNIVERSITY OF MIAMI, Coral Gables, FL (US)

(21) Appl. No.: 13/112,341

(22) Filed: May 20, 2011

Related U.S. Application Data
(60) Provisional application No. 61/347,336, filed on May 21, 2010.

(51) Int. Cl. A61K 39/00; A61P 35/00

(52) U.S. Cl. 424/277.1

(57) ABSTRACT

A cell-based vaccine prolongs the survival of cancer patients. The vaccine includes a dose of irradiated cultured lung adenocarcinoma cells (AD100) transfected with HLA A1 and gp96-Ig (human gp96 wherein the endoplasmic reticulum retention signal, KDEL, is replaced with the Fc-portion of human IgG1 and was injected intradermally into patients suffering from advanced, relapsed, or metastatic NSCLC. Administration of the vaccine increased the mean survival time of the patients compared to that of similar patients treated with placebo. Moreover, the immune response of patients to the vaccine (antigen-induced interferon gamma production by T cells) correlated with the survival times.

### CROSS-REFERENCE TO RELATED APPLICATIONS

The present application claims the priority of U.S. provisional patent application Ser. No. 61/347,336 filed on May 21, 2010, which is incorporated herein in its entirety by reference.

### STATEMENT AS TO FEDERALLY SPONSORED RESEARCH

This invention was made with United States government support under grant number P01 CA109094 awarded by the National Institutes of Health. The United States government has certain rights in the invention.

### FIELD OF THE INVENTION

The invention relates generally to the fields of medicine, oncology, and immunology. More particularly, the invention relates to compositions and methods of prolonging the life of non-small cell lung cancer (NSCLC) patients using cell-based vaccines.

### BACKGROUND

Despite many advances, cancer remains one of the leading causes of death and morbidity in developed nations. Although many of the molecular mechanisms of tumorigenesis have now been revealed, standard treatment of most aggressive tumors continues to be surgical resection, chemotherapy, and radiotherapy. While increasingly successful, each of these treatments still causes numerous undesired side effects. Of the many different types of cancer, NSCLC is one of the most common and deadly.

The annual incidence of NSCLC in the United States exceeds 135,000 (out of a total of 170,000 patients with all types of lung cancer). NSCLC, after metastasis or recurrence, is almost uniformly fatal, with a five-year survival of <5%. The annual mortality rate from lung tumors is higher than that from colon, breast, and prostate carcinoma combined. Results of treatment with chemotherapy for NSCLC disease are poor. Phase III trials have typically demonstrated response rates of 15% to 30%, with median survival of less than one year. A recent meta-analysis of clinical studies randomizing metastatic NSCLC patients between best supportive care and chemotherapy concluded that the mean potential gain in survival was only six weeks. Many new drugs and combinations were recently reported in NSCLC but these regimens result in a complete response in <10% of patients and minimal to modest impact on survival. Factors associated with better survival include stage III disease (versus stage 1V), no weight loss, good performance status, normal LDH, fewer metastatic sites, lack of metastases to vital organs such as brain, meninges, bone marrow and liver, and longer interval to recurrence. Clearly, effective therapy requires innovative strategies.

### SUMMARY

The present invention is related to the discovery that a cell-based vaccine can prolong the survival of cancer patients and reduce progression of the disease. In making this discovery, a vaccine including a dose of cultured lung adenocarcinoma cells (AD100) transfected with HLA A1 and gp96-Ig (human gp96 wherein the endoplasmic reticulum retention signal, KDEL, is replaced with the Fc-portion of human IgG1) were irradiated and injected intradermally into patients suffering from advanced, relapsed, or metastatic NSCLC. The results showed that administration of the vaccine increased the mean survival time of the patients compared to that of similar patients treated with placebo. Moreover, the immune response of patients to the vaccine correlated with the survival times.

Accordingly, in one aspect the invention features a method of treating a cancer in a human subject. This method includes a step of administering the subject a vaccine including a plurality of host cells, each of the host cells co-expressing at least one tumor antigen and a heat shock protein modified to be secreted from each of the host cells. In the method, the survival time of the subject can be increased over the expected survival time for other subject having the same type and stage of cancer. The method might additional include the step of analyzing CD8 T lymphocytes in the blood of the subject both before and/or after administration of the vaccine.

The host cells can be cancer cells (e.g., a cell line originating from the same type and/or grade as the cancer in the subject). Where the cancer in the human subject is a lung cancer, the host cells can be lung cancer cells. As one example, where the lung cancer is non-small cell lung cancer and the host cells can be non-small cell lung cancer cells. The host cells can be from the subject or allogeneic to the subject, and can be irradiated before administration of the vaccine (e.g., to prevent the cells from replicating while allowing heat shock protein secretion to occur for a few to several days after administration). The vaccine can be administered intradermally. In one example, the vaccine is administered at multiple sites in the subject's skin within one day.

Another aspect of the invention is use of a vaccine including a plurality of host cells to treat cancer in a human subject, wherein each of the host cells co-expresses at least one tumor antigen and a heat shock protein modified to be secreted from each of the host cells. In this use, the survival time of the subject can be increased over the expected survival time for other subjects having the same type and stage of cancer. Where the cancer in the human subject is a lung cancer, the host cells can be lung cancer cells. As one example, where the lung cancer is non-small cell lung cancer and the host cells can be non-small cell lung cancer cells. The host cells can be from the subject or allogeneic to the subject, and can be irradiated before administration of the vaccine (e.g., to prevent the cells from replicating while allowing heat shock protein secretion to occur for a few to several days after administration). And the vaccine can be administered intradermally, e.g., at multiple sites in the subject's skin within one day.

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994.

Although methods and materials similar or equivalent to those described herein can be used in the practice or testing of the present invention, suitable methods and materials are described below. All patent documents and publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a graph of Kaplan-Meier curves showing the patient survival data from the clinical study overlaid on historical data from another study. Tick marks indicate patients that were living at the indicated time point.

FIG. 2 is a graph showing the correlation between IFNγ production by peripheral blood CD8+ T cells in response to AD100 cells and overall survival.

FIG. 3 is a series of graphs showing CD8 CTL frequencies detected in IFN-γ ELlspots (left), frequencies of FoxP3(+) CD4 cell in blood (middle), and median survival (right).

### DETAILED DESCRIPTION

The invention encompasses methods and compositions relating to treating cancer. The below described preferred embodiments illustrate adaptation of these compositions and methods. Nonetheless, from the description of these embodiments, other aspects of the invention can be made and/or practiced based on the description provided below.

### Biological Methods

Methods involving conventional immunological and molecular biological techniques are described herein. Immunological methods are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Cell culture techniques are generally known in the art and are described in detail in methodology treatises such as Culture of Animal Cells: A Manual of Basic Technique, 4th edition, by R Ian Freshney, Wiley-Liss, Hoboken, N.J., 2000; and General Techniques of Cell Culture, by Maureen A Harrison and Ian F Rae, Cambridge University Press, Cambridge, UK, 1994. Methods of protein purification are discussed in Guide to Protein Purification: Methods in Enzymology, Vol. 182, Deutscher M P, ed., Academic Press, San Diego, Calif., 1990. General methods of medical treatment are described in McPhee and Papadakis, Current Medical Diagnosis and Treatment 2010, 49^{th} Edition, McGraw-Hill Medical, 2010; and Fauci et al., Harrison's Principles of Internal Medicine, 17^{th} Edition, McGraw-Hill Professional, 2008.

### Treatment of Neoplastic Diseases

The compositions and methods described herein are useful for treating a neoplastic disease (e.g., cancer) in a human subject by administering to the subject a pharmaceutical composition including cells expressing one or more tumor-associated antigens and secreting a heat shock protein (e.g., a secreted form of gp96). The human subject might be male, female, adults, children, seniors (65 and older), and those with other diseases. Particularly preferred subjects are those whose disease has progressed after treatment with chemotherapy, radiotherapy, surgery, and/or biologic agents. Any type of a cancer susceptible to treatment with the vaccines described herein might be targeted, although this technology is thought to be particularly effective (compared to current treatment modalities) for treating cancers originating from lung tissue (e.g., NSCLC). Other types of cancer include cancers originating in the bladder, breast, colon, rectum, endometrium, cervix, kidney, blood (e.g., leukemias and lymphomas), skin (e.g., melanoma), pancreas, prostate, thyroid, testis and ovaries.

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., T0, T is, or T1-4), state of metastasis (e.g., M0, M1), number of observable tumors, node involvement (e.g., N0, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measureable by percent, can be at least 5, 10, 15, 20, 25, 30, 40, 50, 60, 70, 80, or 90% (e.g., survival, or volume or linear dimensions of a tumor).

### Vaccine Compositions

The invention includes pharmaceutical compositions and medicaments that include or use as an active ingredient cells expressing one or more tumor-associated antigens and secreting a heat shock protein (e.g., a secreted form of gp96). The cells might be from one or more human tumor cell lines developed from tumors explanted from a patient (e.g., a single tumor cell line, or multiple tumor cell lines of the same cancer type or different cancer types), or might be a human cell line (e.g., HEK293) not derived from a cancer, but engineered to express one or more tumor-associated antigen. The cells may be irradiated to prevent their replication, while allowing the heat shock protein to be secreted for at least 1, 2, 3, 4, 5, 6, or 7 days (e.g., with at least 2000; 4000; 6000; 8000, 10,000; or 12,000 rad). They may also be engineered to express another marker (e.g., an human MHC protein). The cells for use in the vaccine might be stored frozen and reconstituted just before use in a sterile, pharmaceutically acceptable liquid such as USP grade saline or a buffered salt solution. A list of pharmaceutically acceptable carriers, as well as pharmaceutical formulations, can be found in Remington's Pharmaceutical Sciences, a standard text in this field, and in USP/NF. Other substances may be added to the compositions (e.g., human serum albumin and/or DMSO) and other steps taken to stabilize and/or preserve the compositions, and/or to facilitate their administration to a subject.

### Vaccine Administration

The compositions of the invention may be administered to animals or humans by any suitable technique. Typically, such administration will be parenteral (e.g., intradermal, subcutaneous, intramuscular, or intraperitoneal introduction). In embodiments where the compositions are administered by injection, the needle size should be selected to minimize shear to protect the integrity of the cells (e.g., depending on the application, larger than 14, 16, 18, 20, 22, or 24 gauge). The compositions are preferably administered in multiple injections (e.g., at least 2, 3, 4, 5, 6, 7, 8, 9, 10, 12, 14, 16, 18, 20, 25, 30, 40, 45, or 50 injections) or by continuous infusion (e.g., using a pump) at multiple sites (e.g., at least 2, 3, 4, 5, 6, 7, 8, 9, 10, 12, or 14 sites).

In one example, cutaneous injections are performed at multiple body sites to reduce extent of local skin reactions. On a given vaccination day, the patient receives the assigned total dose of cells administered from one syringe in 3 to 5 separate intradermal injections of the dose (e.g., at least 0.4 ml, 0.2 ml, or 0.1 ml) each in an extremity spaced at least about 5 cm (e.g., at least 4.5, 5, 6, 7, 8, 9, or cm) at needle entry from the nearest neighboring injection. On subsequent vaccination days, the injection sites are rotated to different limbs in a clockwise or counter-clockwise manner.

### Dose and Number of Vaccinations

A therapeutically effective amount is an amount which is capable of producing a medically desirable result in a treated animal or human. An effective amount of the compositions of the invention is an amount which shows clinical efficacy in patients as measured by an increase in expected survival (compared to the mean of similar patients) or the improvement in one or more of the cancer characteristics described above. As is well known in the medical arts, dosage for any one animal or human depends on many factors, including the subject's size, body surface area, age, the particular composition to be administered, sex, time and route of administration, general health, and other drugs being administered concurrently. Preferred doses per administration are those number of cells that secrete at least 100, 500, 1000, 2000, 3000, 4000, 5000, 6000, 7000, 8000, or 9000 mg/ml/day of the secreted from of heat shock protein in in vitro culture. The number of cells in each dose may range from 100,000 to 100,000,000 (e.g., about 100,000; 250,000; 500,000; 750,000; 1,000,000; 2,000,000; 5,000,000; 10,000,000; 20,000,000; 50,000,000; or 100,000,000+1-20, 10, or 5%) The dose may be given repeatedly, e.g., hourly, daily, semi-weekly, weekly, bi-weekly, tri-weekly, or monthly.

As an example of one administration protocol, on each visit for therapy (every week or every other week) a clinical evaluation of the cancer and of toxicity is conducted. Blood samples for immunological evaluation are obtained on Day 1 of each course before vaccination is given. Patients with evidence of stable disease or responding NSCLC, and acceptable toxicity (autoimmune <grade 2, and grade ≦2 for other body systems) upon completion of the first course of vaccination are treated with an additional course at the same dose and schedule. A third course at the same dose and schedule is given provided that the patient has evidence of stable disease or responding NSCLC, and acceptable toxicity (autoimmune <grade 2, and grade ≦2 for other body systems) on completion of the second course.

### EXAMPLES

### Example I

### Drug

Name: Ad100-gp96Ig-HLA A1, short gp96-vaccine (gp96-Ig and HLA A1 transfected NSCLC cell line). This drug was described in U.S. patent application Ser. No. 11/878,460. A human lung adenocarcinoma cell line was established in 1994 from a biopsy of a lung cancer patient and is designated as Ad#100. The patient was a 74-year-old white male who in 1993 was presented with initial symptoms of pelvic pain due to bone erosion of the iliac crest and lung nodules of the primary and metastatic pulmonary adenocarcinoma. Cancer cells for culture were obtained by bone marrow aspiration from the area of pelvic bone destruction. The patient was treated with radiation therapy to the pelvis, but expired one month after diagnosis. The cell line derived from this patient has been kept in culture in standard medium (described below) and is free of contamination by mycoplasma, virus or other adventitious agents. The cell line is homogeneous, adherent to plastic, and grows with a rate of division of approximately 26 h. The cell line has been tested and determined to be free of the following: HIV-1, HIV-2, HTLV-1, HTLV-2, HBV, Adenovirus, Polyomavirus, CMV, EBV, HHV6, HCV, VZV, Parvovirus B19, HPV, and Mycoplasma.

Ad100 was transfected with the plasmid cDNA 1345-neo-gp96Ig-HLA A1′ and selected with G418. B45 is a vector derived form the bovine papilloma virus by deletion of the capsid-encoding genes L1 and L2 and by further deletion of the potentially transforming genes E5, E6 and E7. The vector contains two cassettes for expression of eukariotic cDNAs; in this case HLA A1 driven by the metallothionein promoter and gp96-Ig driven by the cytomegalo-virus (CMV) promoter. The shuttle vector also contains the β-lactamase gene for selection in E. coli and the neomycin-resistance gene under the thymidine kinase promoter for G418 selection of transfected Ad100 cells. The E1 and E2 gene of the B45 vector encode the two viral proteins that are required for episomal replication of the plasmid and high level expression of the encoded cDNAs. High level expression of cDNA's is further enhanced by inclusion of a non-coding portion of the human β-globin gene. The vaccine cell line is permanently transfected (no new transfections are necessary) and maintained under periodic reselection conditions in G418 to ensure maintenance of the plasmid-episome in transfected cells.

Expression of human HLA A1 was determined by FACS analysis using specific antibodies. Preparations expressing HLA A1 on 70% or more cells were used for vaccination. Expression of gp96-Ig was measured by an enzyme linked immuno-sorbent assay (ELISA) detecting the Ig-portion of the gp96-Ig fusion protein. Cells producing ≧60 ng of gp96-Ig in 24 hours by 10⁶ cells were used for vaccination.

The cell line was expanded in a GMP facility under sterile conditions. Absence of bacterial, viral, yeast, and mycoplasma and levels of endotoxin was determined for each batch by FDA mandated and approved assays.

FCS, IMDM, trypsin EDTA, HBSS, G418 were obtained from GIBCO and were certified free of adventitious reagents. DMSO was from Sigma and also free of adventitious agents. Human serum albumin and buffered saline solution were pharmaceutical grade. Batches of cells were expanded to about 1-5×10⁹ cells in tissue culture flasks, and tested for presence of expression HLA A1 by FACS and gp96Ig by ELISA. Cells were harvested, washed, and re-suspended in buffered saline+10% DMSO+0.5% human serum albumin at 4° C., aliquoted to 5×10⁷/0.5 ml and irradiated at 12,000rad using a Cobalt-irradiator at 4° C. Samples were withdrawn for biological and safety analysis. The remaining aliquots were frozen and stored at −135° C.

To insure that the vaccine cell line after irradiation was replication incompetent but maintained biological activity, the AD100-gp96-Ig-HLA A1 cell line after 12,000 rad irradiation was tested as follows: Colony formation in soft agar: No detectable colonies from 10⁸ cells irradiated cells plated; Gp96-Ig secretion: approaches 0 ng after 14 days following radiation while unirradiated controls maintain gp96-Ig production; Thymidine incorporation is increased in irradiated cells for the first 48 h (compared to controls), due to DNA repair (after one week irradiated cells show no thymidine uptake in contrast to control cells that continue to proliferate and take up thymidine); and the Cobalt irradiator is calibrated at set up and annual adjustments for decay. The Cobalt irradiator is a panoramic irradiator; radiation dose depends solely on physical decay of the source which is adjusted annually.

The vaccine to be injected contains irradiated Ad100 cells expressing HLA A1 on at least 70% of the cells and produce ≧60 ng gp96-Ig/24 h×1 million cells; ≧70% viability by trypan blue exclusion. The cells are resuspended in buffered saline with 0.5% human serum albumin, 10% DMSO.

### Example II

### CD8 Response

CD8 cells are purified from 15 ml blood with the Rosette-Sep kit from Stem Cell Technologies (Vancouver, Canada). This procedure generates about 1.5 million CD8 cells of about 85% purity by negative selection, eliminating also NK cells with anti CD56. Primary contaminating cells are B cells. CD8 cells (20,000) are challenged in triplicate for 48 h in ELI-spot plates with 1,000 cells each of autologous tumor cells, AD100-HLA A1-gp96Ig (vaccine), AD100 (untransfected), MeI-A1 (HLA A1 transfected melanoma), SCLC-A1 (A1 transfected small cell lung carcinoma), K562 (NK target) and no challenge. Secretion of IFN-γ, of IL-4 and of granzyme B is determined using the appropriate ELI-spot antibodies (Becton & Dickinson). Samples are run in triplicate and are quantitated in an automated ELI-spot reader from C.T.L (Cellular Technologies Ltd, Cleveland Ohio).

### Example III

### Clinical Outcome

Progression-free survival and overall survival are estimated by the Kaplan-Meier method, stratified by dose-schedule cohort. The corresponding median survival times (with 90% confidence limits) are determined, as is the cumulative percentage of patients remaining alive at 6, 12, 18, 24, and 36 months post enrollment. To the extent possible, proportional hazards regression analysis is used to assess progression-free survival and overall survival in relation to dose-schedule assignment, treatment received (e.g., total dose, number of vaccinations), baseline characteristics, and various measures of immune response (e.g., CD8 fold increase).

### Example IV

### A Phase I Study of Patients with Non-Small Cell Lung Cancer (NSCLC), Stage IIIB/IV, with Multiple Pre-Treatment Regimens

The characteristics defining the enrolled patient population were: locally advanced or metastatic stage IIIB/IV NSCLC, ECOG performance status 0-2, and multiple pre-treatments including chemotherapy, radiotherapy and biologic modifier therapy. Patients are placed in one of three arms. Patients enrolled in Arm 1 receive 9 bi-weekly doses of AD100-gp96-A1, patients enrolled in Arm 2 receive 18 once-weekly doses of AD100-gp96-A1, and patients enrolled in Arm 3 receive 36 twice-weekly doses of AD100-gp96-A1. The total dose of AD100-gp96-A1 is constant over the course of treatment for each of the 3 arms. No additional adjuvants or therapies are given concurrent with AD100-gp96-A1 therapy.

### Example V

### Results at One Time Period

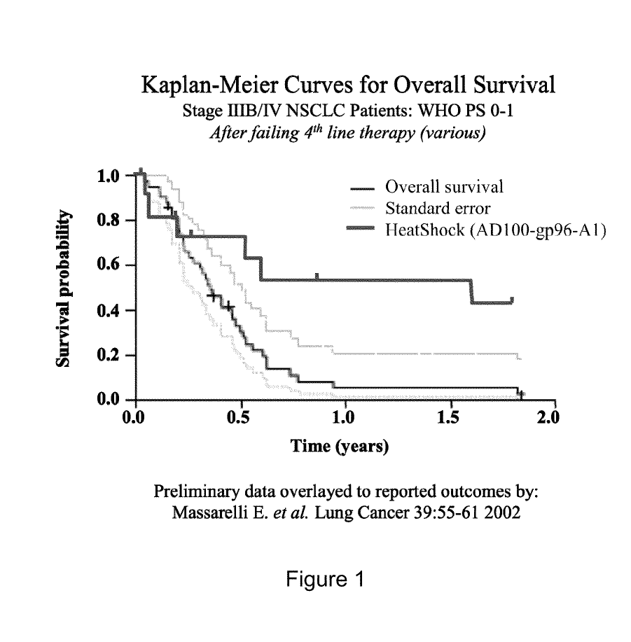

Of the first 12 patients enrolled in the study, one passed away before receiving the vaccine, nine were enrolled in Arm 1 (bi-weekly doses), one was enrolled in Arm 2 (once-weekly doses), and one was enrolled in Arm 3 (twice-weekly doses). No vaccine-related serious adverse events (SAE) were reported, while all patients experienced vaccine-site reactions including erythema and minor swelling. One patient passed away within a month of receiving the vaccine, however the SAE report for this patient determined that the death was due to the progression of the disease, and not from the vaccine treatment. Overall survival of the 12 patients that have received at least one dose of the vaccine is plotted in FIG. 1.

FIG. 1 overlays the study data with historical data from the Massarelli study (Lung Cancer 39:55061, 2002). The Massarelli study is an excellent comparator for this study because it is one of the only studies to break down patient survival and responses according to the number of prior therapies they have received. The Massarelli study provides survival data for patients that have progressed through 4 lines of therapy. The patients from which FIG. 1 data was derived averaged failing 5.3 lines of therapy prior to treatment with AD 100-gp96-A1 (median 4 lines, surgery and radiotherapy not included).

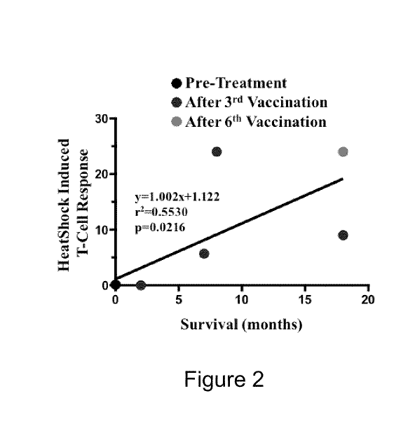

To evaluate patient immune responses, peripheral blood samples were drawn from each patient before receiving the vaccine and subsequently at 6-week intervals (between each ‘course’ of treatment). Peripheral blood lymphocytes were then evaluated for production of cytokines such as interferon-gamma and granzyme B in response to stimulation with AD100 vaccine cells or other unrelated cell lines. The lymphocyte subset composition of the peripheral blood was also analyzed by flow cytometry. Referring to FIG. 2, data collected from 4 patients enrolled in Arm 1 demonstrate a correlation between the production of interferon γ by CD8+ T cells and overall survival.

Several patients achieved disease stabilization without completing a full course of treatment. One patient survived over 20 months post-treatment at this time point; a second patient survived 18 months post-treatment. The magnitude of the AD100-gp96-A1-specific T cell response measured during therapy appears to be predictive of increased survival.

Patient 1003 had locally advanced and progressive disease at the time of trial enrollment with significant pleural effusion. Patient 1006 had a large, diffuse mass throughout one lung that was locally invasive to the carina. These two patients fall into the T4-pleural effusion and T4-invasive subtypes of stage IIIB/IV NSCLC. The largest study performed to date comparing the relative survival of stage IIIB/IV NSCLC with each of the various sub-types (William W N et al, Chest 2009; 136) determined that the only sub-type of stage IIIB/IV NSCLC with improved overall survival were patients with T4-satellite disease. Patients with T4-invasive or -pleural effusion were found to have overall survival indistinguishable from patients with stage IV disease. Thus, there is no evidence to suggest that these patients had disease with an improved overall prognosis at the time of enrollment compared to other patients enrolled in the trial. In addition, there does not seem to be a clear correlation between the number and site of positive lymph nodes or metastatic lesions and overall survival for this small group of patients. Patient 1011, whom despite having the most advanced disease of any of the enrolled patients, has achieved stable disease and almost completed all three courses of therapy.

### Example VI

### Results at a Later Time Period

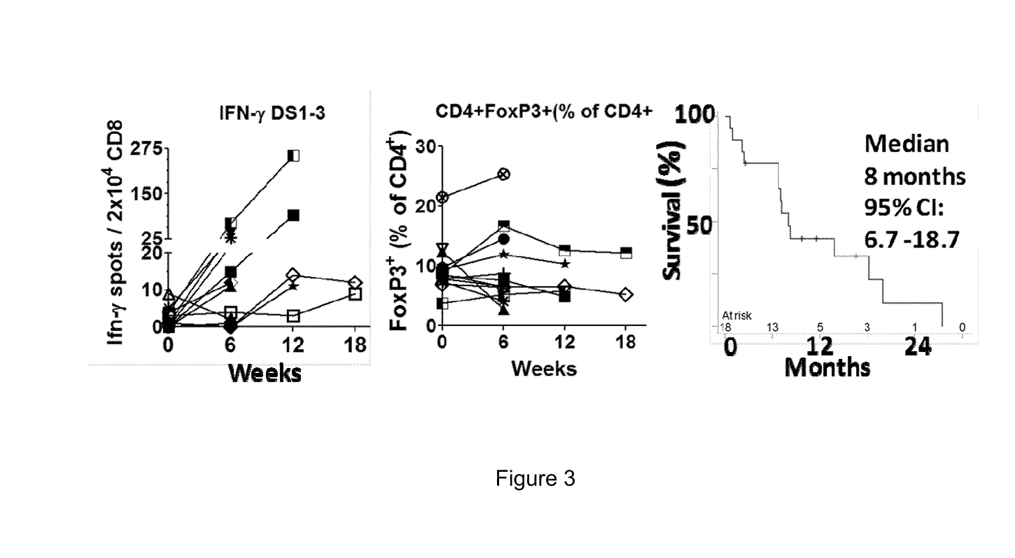

Nineteen patients have been enrolled and vaccinated over an 18 week period with a total of 4.5×10⁸ vaccine cells in three dose administration schedules: dose schedule (DS-1) 9 vaccinations (each 5×10⁷ cells) every 2 weeks (Arm 1); dose schedule (DS-2) 18 vaccinations (each 2.5×10⁷ cells) every week (Arm 2); and dose schedule (DS-3) 36 vaccinations (each 1.25×10⁷ cells), twice per week (Arm 3) Immune response and clinical response was measured at baseline, after 6, 12, and 18 weeks. The vaccine was well tolerated at all dose schedules, with minimal, expected side effects at the vaccination site. It generated significant CD8 CTL frequencies detected in IFN-γ ELI spots (FIG. 3, left) and, in some patients, a trend to reduced frequencies of FoxP3(+) CD4 cell in blood (FIG. 3, middle). Median survival was estimated as 8.0 months (95% CI: 6.7 to 18.2) which is twice the expected survival estimate (FIG. 3, right). Although the comparison was limited due to incomplete accrual to DS-2 and DS-3, two of four patients in DS-2 (weekly vaccination) as well as two of three patients in DS 3 (twice weekly vaccination) have survived longer than the median survival time of 7.1 months for the 11 patients in DS 1. Specifically, there were two patients who died at 8.3 and 20 months (DS-2 and DS-3, respectively) and two patients who were alive at 9.7 and 11.5 months (DS-3 and DS-2, respectively).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a cancer in a human subject, the method comprising a step of administering the subject a vaccine comprising a plurality of host cells, each of the host cells co-expressing at least one tumor antigen and a heat shock protein modified to be secreted from each of the host cells.

2. The method of claim 1, wherein the survival time of the subject is increased over the expected survival time for other subject having the same type and stage of cancer.

3. The method of claim 1, further comprising the step of analyzing CD8 T lymphocytes in the blood of the subject both before and after administration of the vaccine.

4. The method of claim 1, wherein the host cell is a cancer cell.

5. The method of claim 4, wherein the cancer in the human subject is a lung cancer and the host cells are lung cancer cells.

6. The method of claim 5, wherein the lung cancer is non-small cell lung cancer and the host cells are non-small cell lung cancer cells.

7. The method of claim 1, wherein the host cells are allogeneic to the subject.

8. The method of claim 1, wherein the host cells are irradiated before administration of the vaccine.

9. The method of claim 1, wherein the vaccine is administered intradermally.

10. The method of claim 1, wherein the vaccine is administered at multiple sites in the subject's skin within one day.
